(19) United States
(12) Patent Application Publication
(10) Pub. No.: US 2024/0350814 A1
(43) Pub. Date: Oct. 24, 2024

(54) METHOD FOR PREPARING AN IMPLANTABLE DEVICE

(71) Applicant: Hylomorph AG, Zürich (CH)

(72) Inventors: Aldo FERRARI, Zürich (CH); Simone BOTTAN, Zürich (CH); Francesco ROBOTTI, Zürich (CH); Georgios STEFOPOULOS, Zürich (CH)

(73) Assignee: Hylomorph AG, Zürich (CH)

(21) Appl. No.: 18/574,984

(22) PCT Filed: Jun. 7, 2022
(86) PCT No.: PCT/EP2022/065433

§ 371 Date: Dec. 28, 2023

(30) Foreign Application Priority Data
Jun. 29, 2021 (EP) 21182425.5

(51) Int. Cl. A61N 1/375; A61L 31/04; A61L 31/16

(52) U.S. Cl. CPC A61N 1/37512; A61L 31/044; A61L 31/16; A61L 2300/406

(57) ABSTRACT

Method for the preparation of an implantable device, wherein in a first step a non-degradable implantable device is at least partially covered or surrounded by at least one separate layer of material comprising a therapeutically active substance providing for sustained controlled release of said substance under physiological conditions, and wherein in a second step this is inserted into a wet or humid pouch through an opening thereof.

### TECHNICAL FIELD

The present invention relates to methods for preparing implantable devices as well as kits of part for such methods, elements for such methods and methods for applying implantable devices.

### PRIOR ART

Non-degradable implantable devices, implanted to stay in place, need to be structured to avoid a negative foreign body response of the patient. This applies to fully embedded implants such as breast implants or pacemakers, but also implants that require long-term trans dermal communication. Certain types of implantable medical devices require long-term trans dermal communication between the internal and external portions of the device.

Examples of such devices include left ventricular assist devices, tissue expanders, arteriovenous shunts, and gastric lap bands. The transdermal portion of the device can be in the form of a catheter, a gas insufflation tube, or one or several electrical wires. These types of transdermal medical devices are susceptible to infection.

In order to increase the compatibility between the surrounding tissue and the actual implantable medical device it is possible to provide for a pouch of a material into which the implantable medical device is put before insertion into the body, providing an optimum interface between the surrounding tissue and the implant avoiding complications and assisting healing in of the device.

A problem with implantable medical devices, in particular those with long-term transdermal communication, is that there is a high infection rate after implantation. Typically, this is treated by oral or intravenous administration of antibiotics, alone or combined with local flushing with antibiotic solution, however in particular in case of implants located at physiologically delicate places in the body which at the same time are not quickly accessible for orally administered antibiotics, this may not be sufficient.

There remains a need to reduce the incidence of infection associated with implantable devices.

Wound dressings are designed to support the wounded region, protect it from infection, and, in certain cases, actively promote wound healing by creating a favorable environment for cell growth.

The response to wounding, defined as a breakage of bodily tissue, involves an inflammation phase, a migratory phase and a remodeling phase. The inflammation phase is the acute response to a wound and its purpose is to quickly seal the wound and produce chemical factors that employ cells to migrate into the wound and start the wound healing process.

During the migratory phase, cells rapidly migrate into the wound and start laying down provisional extracellular matrix that will be the base of the healed tissue. During the remodeling stage, the newly created tissue slowly matures into its permanent form.

Standard wound dressings facilitate wound healing by: 1. mechanically holding the wound edges together to allow easier cell migration; 2. mechanically sealing the wound to prevent contamination by pathogens; 3. in some advanced dressings providing an environment that actively promotes faster wound healing, usually by exposing the wounded tissue to a hydrated gel. Improved materials for these applications would be desirable.

In plastic surgery commercial silicone implants (e.g. breasts, calf, buttock, chest, biceps), but also the above-mentioned implantable medical devices often fail due to foreign body reaction and scar-tissue encapsulation. Also for such applications improved materials/coatings would be desirable.

In cosmetics disposable flat and unstructured cellulose masks are available (e.g. face, hands and feet) as masks that enhance skin hydration, as well as the absorption of metabolic waste products and the release of nutrients or other compounds to the skins.

WO2015040106A1 proposes a method for the self-assembled production of a topographically surface structured cellulose element which can be used as a material for such a pouch, wherein in a first step a mold with on one side a first surface which is in a complementary manner topographically surface structured and which is permeable to oxygen is provided, wherein a liquid growth medium containing cellulose producing bacteria is provided, and wherein the mold is placed to form a liquid/air interface of the liquid growth medium such that the side of the mold with the first surface is in direct contact with the liquid growth medium, and with an opposite side is facing air or a specifically provided oxygen containing gas surrounding, allowing for said bacteria to produce and deposit cellulose on said first surface and developing on the interface therewith a topographically surface structured surface complementary thereto, until a contiguous cellulose layer with a thickness of the element of at least 0.3 mm is formed; and wherein in a second step the element is removed from said mold. Furthermore, the invention relates to elements made using such a method and uses of such elements for various applications. The corresponding pouch provides for optimum compatibility to the surrounding tissue directly after implantation but also over longer time spans when the implant is to remain in the body.

US-A-2019009063 discloses an inflatable balloon which is enclosed by an expandable cover which becomes increasingly porous/permeable during expansion. The balloon is coated or enclosed with a matrix which contains a pharmaceutically active agent. During expansion of the balloon, the pharmaceutically active agent is released or extruded through the expandable cover into a body cavity such as an artery or vein.

US-A-2007141106 provides a medical system for the administration of a pharmaceutical agent in vivo to a patient. The medical system includes a medical implant positionable in a body of a patient. A pharmaceutical agent is disposed on the medical implant and at least partially coated with a reactive coating. The reactive coating acts to control the release of the pharmaceutical agent. An energy unit is provided for transmitting an energy signal to the reactive coating, wherein the reactive coating reacts to the energy signal to increase the release rate of the pharmaceutical agent.

WO-A-2008136856 describes biodegradable and resorbable polymer pouches for use with cardiac rhythm management devices (CRMs) and other implantable medical devices (IMDs), i.e., a pouch, covering, or other receptacle capable of encasing, surrounding and/or holding the CRM or other IMD for the purpose of securing it in position, inhibiting or reducing bacterial growth, providing pain relief and/or inhibiting scarring or fibrosis on or around the CRM or other IMD. Optionally, the biodegradable and resorbable pouches of the invention include one or more drugs in the polymer matrix to provide prophylactic effects and alleviate side effects or complications associated with the surgery or implantation of the CRM or other IMD.

US-A-2020197712 describes nonwoven resorbable pouches that at least partially enclose implantable medical devices and improved methods for producing the implantable medical device pouches. The nonwoven pouches may comprise one or more drugs. Implantable medical devices that are placed in the pouches prior to implantation are prevented from migrating from the site of implantation by tissue ingrowth into the pouch. Antibiotics may be incorporated into the pouches to prevent post-operative infections. The pouches may be formed in fewer steps than conventional pouches, and without polymer coatings. Nonwoven pouches can be formed in one step by dry spinning instead of using multiple processing steps. In embodiments, the nonwoven pouches are smoother on the inside than the outside to tightly fit the implantable medical devices internally while encouraging external tissue ingrowth. In embodiments, the nonwoven pouches eliminate the use of knitted or woven multifilament fibers that can trap bacteria and result in post-operative infection.

### SUMMARY OF THE INVENTION

It is therefore an object of the present invention to provide for improved elements, in particular cellulose elements, for encapsulating implants to be implanted.

The present invention relates to a method claimed, a kit of parts as claimed, an implantable device as claimed as well as uses thereof.

More specifically, according to a first aspect of the present invention, it relates to a method for the preparation of an implantable device.

According to this method, in a first step a non-degradable implantable device is at least partially covered or surrounded by at least one separate layer of material comprising a therapeutically active substance providing for (preferably sustained controlled) release of said substance under physiological conditions.

Typically, the separate layer is provided in the form of a typically flat sheet of thin material in which the therapeutically active substance is embedded in a way providing for sustained controlled release. The separate sheet can be provided as a contiguous layer, however it may also be a perforated layer or take the form of a layer in the form of a grid or mesh or nonwoven. It is also possible that this separate layer is provided as a layer having a coating of the therapeutically active substance providing for sustained controlled release. In particular in case of a flat non-degradable implantable device, such a sheet is applied on both sides having the largest surface of such a device. If the device is cylindrical or the like, it may also be possible to roll a corresponding separate layer around the device. The separate layer may just be loosely assembled with the non-degradable implantable device, it may however also be attached to the non-degradable implantable device in this first step, this may take place by electrostatic attraction, by capillary forces, by actual gluing with an appropriate glue, or by mechanical attachment, e.g. by providing a clamping or by providing a crimping or similar means. Preferably the separate layer is provided around the non-degradable implantable device so that in the implanted state the majority of the inside of the pouch is in contact with such separate layers so that there is diffusion of the therapeutically active substance through the material of the pouch over the majority of the surface of the pouch.

According to the invention, in a second step this, i.e. the assembly of the non-degradable implantable device with the at least one separate layer, is inserted into a wet or humid cellulose pouch through an opening thereof. More generally speaking, it is inserted into a wet or humid self-supporting pouch of at least one of hydrogel, cellulose or collagen through an opening thereof, preferably the pouch is a collagen or cellulose pouch. The pouch in the sense here, like an envelope, provides at least one, preferably essentially contiguous, layer of material forming an enclosure essentially fully enclosing the non-degradable implantable device when located inside the pouch, closed in all directions except for an opening, on one side or two sides of the pouch only, allowing for insertion of the non-degradable implantable device. In case of cellulose, this is not elastic, therefore not expandable. It cannot be used for an inflatable balloon, in any circumstance, even for a minimal expansion, as it would rupture.

In fact, the humid pouch of at least one of self-supporting hydrogel, cellulose or collagen is nonelastic, which can preferably be quantified in that after applying and releasing tension in loading and unloading stress-strain curves with even a low stress (stress values of σ as low as 0.005, 0.01 of 0.5 MPa, or in the range of 0.01-0.1 MPa), the material of the pouch is unable to go back to its initial strain-free state.

The cellulose layer of the pouch can be provided as non-degradable pouch, therefore accompanying the non-degradable implantable device for its entire lifetime. In the alternative, the cellulose layer can be provided as degradable element which is resorbed by the body in a certain amount of time.

Key elements of the present invention can be as follows:

The therapeutically active substance, in particular in the form of antibiotic shall be provided in an efficient way so as to avoid post implantation infections and the like. A cellulose pouch which is suitable and adapted to stay in place after the implantation and to remain around the non-degradable implantable device is typically produced in a biochemical process involving living organisms, so incorporating a corresponding therapeutically active substance into the cellulose pouch material is not possible, in particular not for antibiotics. Further, the cellulose pouch material cannot be prepared and then provided as a dry layer for many practical reasons, related to the storage and packaging, preparation and use. Also impregnation or coating of the pouch material after the making process with therapeutically active substance is not possible, since the wet or humid cellulose will suffer from the presence of the antibiotic or another therapeutically active substance during storage, and since the direct presence of such a material on the wet or humid cellulose pouch material will change the properties thereof, and coatings on the wet cellulose would not adhere.

Further most therapeutically active substances cannot be stored under wet or humid conditions without significantly impairing storage time for degradation reasons.

The proposed approach therefore provides for a very simple and versatile solution to post implantation infections and the like. The proposed method is independent of the non-degradable implantable device, i.e. it can be combined with any kind of non-degradable implantable device.

The cellulose pouch is porous and open for diffusion of the therapeutically active substance through the material of the pouch. The cellulose pouch not only allows for penetration of the therapeutically active substance, it also distributes the therapeutically active substance over its surface, thereby homogenizing the release of the therapeutically active substance essentially over the full surface even if not the full inner surface of the pouch is covered by the separate layers.

This is a novel approach different from what is in the prior art. In US-A-2019009063 there is a matrix which contains a pharmaceutically active agent. During expansion of the balloon, the pharmaceutically active agent is released or extruded through the expandable cover into a body cavity such as an artery or vein. In the present approach, there is no expansion necessary for the release of active agents. In fact the sustained release only requires that the film containing the molecules (material comprising a therapeutically active substance) is releasing them only upon contact with the liquid, so in particular the water in the cellulose pouch. The cellulose matrix of the pouch is not releasing the antibiotic molecules, it is in fact doing rather the opposite, that is, it is retarding their diffusion acting as diffusion barrier. The release is therefore essentially purely regulated by the contact of the inner film with the liquid environment. The release of the active molecules into the body cavities is passively controlled and in fact retarded (so not promoted) by the cellulose matrix porosity of the pouch. In this sense the proposed matrix of the pouch has an opposite function that the one described in US-A-2019009063 and in further prior art approaches.

The size of the layers, their content of the therapeutically active substance and composition of the therapeutically active substance can be adapted and tailored to the specific needs on an individual basis. The separate layers provide for a clearly defined dosage of the therapeutically active substance which allows to comply with regulatory requirements and which provides for a control dosage over the release time.

Typically the bacteria causing problems in the implantation process are normally located on the surface of the implantable device. By locating the layers right on that surface the antibiotic is exactly located where it needs to act, and its dosage is highly controlled contrary to for example dip coating processes.

According to a first preferred embodiment, the wet or humid cellulose pouch has a water content in the range of 50-98%, preferably in the range of 90-98%.

According to a further preferred embodiment, the wet or humid cellulose pouch has a diffusivity or rather a diffusion constant or mass diffusivity for the therapeutically active substance in water in the range of 10⁻¹¹ to 10⁻¹⁰ m²/s, preferably in the range of 10⁻¹⁰ to 9*10⁻¹⁰ m²/s, normally at a temperature of 37° C., wherein preferably these values are given for a cellulose pouch. In practice, the values are determined for the effective diffusivity for the therapeutically active substance (e.g. Minocycline and Rifampin) across a cellulose layer and measured in water at 37 degrees, as compared with deviation from free diffusion in water for the same molecules. This deviation is introduced by the porosity and tortuosity of the porous material.

The wet or humid cellulose pouch typically has a thickness of at least 0.3 mm, preferably in the range of 0.5-10 mm or in the range of 0.5-5 mm.

The wet or humid cellulose pouch may have, at least on its outside surface, a topographical surface structure with a height in the range of 0.5-2 μm, and in case of a groove/ridge topographical structure a periodicity of the structure in the range of 0.5-100 μm and in case of a pillar topographical structure a periodicity of the structure a periodicity at least in one dimension, preferably in three different directions, in the range of 5-50 μm, preferably in the range of 7-15 μm. Preferably, the topological structure is one as described in WO2015040106A1, the content of which for the topological structure as well as for the making of the cellulose pouch material is expressly included into this disclosure.

Accordingly, according to a preferred embodiment the wet or humid cellulose pouch is produced before said first step using self-assembled production of a topographically surface structured cellulose element wherein a mold with on one side a first surface which is in a complementary manner topographically surface structured and which is permeable to oxygen is provided, wherein a liquid growth medium containing cellulose producing bacteria is provided, and wherein the mold is placed to form a liquid/air interface of the liquid growth medium such that the side of the mold with the first surface is in direct contact with the liquid growth medium, and with an opposite side is facing air or a specifically provided oxygen containing gas surrounding, allowing for said bacteria to produce and deposit cellulose on said first surface and developing on the interface therewith a topographically surface structured surface complementary thereto, until a contiguous cellulose layer with a thickness of the element of at least 0.3 mm is formed;

and wherein in a following step the element is removed from said mold, and said pouch is produced from said element.

Preferably, between this production, which may entail actual formation of the pouch with an opening by sealing edges after a folding of a sheet, and the first step the element or the pouch can be stored in a wet environment, preferably in a corresponding suitable and adapted container.

Said separate layer typically comprises, as therapeutically active substance, at least one antibiotic, antiseptic, haemostatic, antinflammatory, chemotherapic, hormones, chemokines or disinfectant or a combination thereof, wherein preferably the therapeutically active substance is selected from at least one antibiotic selected from the group of tetracyclines, penicillins, macrolides, ansamycines, wherein it is preferably selected from the group consisting of Tetracycline, Chlortetracycline, Oxytetracycline, Demeclocycline, Lymecycline, Meclocycline, Methacycline, Minocycline, Rolitetracycline, Doxycycline, Tigecycline, Eravacycline, Sarecycline, Omadacycline, Rifampicine, or a combination thereof, in particular a combination of Minocycline HCl and Rifampicine.

The concentration of the therapeutically active substance in the layer is preferably in the range of 5-50%, preferably in the range of 10-45% or in the range of 25-40%, in each case given as % w/w dry with respect to the total of the layer.

Normally in addition to the therapeutically active substance the layer comprises at least one degradable or non-degradable polymeric material for embedding the therapeutically active substance and for providing a controlled release behavior. Preferably the material is in the form of a degradable polymeric material, preferably selected from the group consisting of polylactic acid, poly(lactic-co-glycolic acid) (PLGA), polyglycolic acid, poly(L-lactide) (PLLA), poly(D.L-lactide), (PLA) polyglycolic acid polyglycolide (PGA), poly(L-lactide-co-D.L-lactide) (PLLA/PLA), poly(D, L-lactide-co-glycolide) (PLA/PGA), poly(glycolide-co-trimethylene carbonate) (PGA/PTMC), poly(D.L-lactide-co-caprolactone) (PLA/PCL), poly(glycolide-co-caprolactone) (PGA/PCL), poly(oxa) esters, polyethylene oxide (PEO), polydioxanone (PDS), polypropylene fumarate, polyethyl glutamate-co-glutamic acid), poly(tert-butyloxy-carbonylmethyl glutamate), poly caprolactone (PCL), polycaprolactone co-butylacrylate, polyhydroxybutyrate (PHBT), polyhydroxybutyrate, poly(phosphacene), poly(phosphate ester), poly(amino acid), polydepsipeptides, polyiminocarbonates, poly(dimethyl-trimethylene carbonate)-co-(trimethylene carbonate), poly(orthoesters), tyrosine-derived polycarbonates, tyrosine-derived polyiminocarbonates, tyrosine-derived polyphosphonates, polyethylene oxide, polyethylene glycol, polyalkylene oxides, and hydroxypropylmethylcellulose polysaccharides including hyaluronic acid, chitosan and regenerate cellulose, and proteins including gelatin and collagen, and mixtures and copolymers thereof.

The separate layer may take the form of a grid, nonwoven or woven or of a contiguous layer. Correspondingly therefore the separate layer may also be constituted by fibers of a material which is coated or impregnated or contains the therapeutically active substance in appropriate concentration.

The separate layer may also take the form of at least one strip, which is wrapped around the non-degradable implant. Furthermore the separate layer may take the form of a pouch of the corresponding material or the form of a stretchable film like a clingfilm to completely wrap the non-degradable implant.

Said layer as preferably provided as at least 2 sheets which are put on opposing main faces of the non-degradable implantable device before insertion into said wet or humid cellulose pouch.

Preferably said separate layer is provided as a dry layer with a water content of less than 5%, preferably less than 2%, more preferably less than 1%, in each case given as % w/w. Said layer (or said plurality of layers in case of each layer) has a thickness in the range of 0.01-3 mm, preferably in the range of 0.02-0.2 mm.

Said non-degradable implantable device can be selected for example from the group of cardiovascular implant and/or device, in particular a pacemaker or cardioverter defibrillator; neurostimulator, neuromodulator, implantable pulse generators, cosmetic implant, preferably in the form of a breast implant, cuff implant, pectoral implant, biceps implant, buttock implant, gluteal implant; orthopedic prosthesis; a sensor and/or electrical stimulation device; draining system, preferably a catheter; pump or tubing system; ophthalmological device; hearing device; bionic device.

Subsequent to the second step in a third step said opening is typically closed, preferably by way of a suture, crimping or gluing, or a combination thereof wherein elements, in particular tubing and/or wiring attached to and connected with said non-degradable implantable device if present remain penetrating said opening.

Further the present invention relates to a kit of parts for use in a method as described above, comprising
at least one wet or humid cellulose pouch having an opening in a first wet package;
and at least one layer, preferably a plurality of layers, of material comprising a therapeutically active substance providing for sustained controlled release of said substance under physiological conditions in a separate wet or dry package, preferably in a dry package.

Typically there is provided at least two layers in said separate package.

Preferably the separate layers take the form of rectangular or quadratic sheets with length and/or width in the range of 50-250 mm, preferably in the range of 60-90 mm, wherein they particularly preferably take the form of a rectangle having a length in the range of 70-110 mm, preferably in the range of 80-90 mm, and a width in the range of 50-90 mm, preferably in the range of 60-80 mm.

Said first package may comprise elements for maintaining controlled humidity, preferably in the form of aluminum plastic laminate pouches, and/or wherein said separate package may comprise elements for maintaining dryness, preferably desiccant elements.

The present invention also relates to an implantable device produced as detailed above, preferably using a kit as detailed above, wherein a non-degradable implantable device at least partially covered or surrounded by at least one separate layer of material comprising a therapeutically active substance providing for sustained controlled release of said substance under physiological conditions, is located in a wet or humid cellulose pouch.

Last but not least the present invention relates to a method of implanting an implantable device as detailed above or ones as produced above in a next step into a human or animal body, wherein said implantable device is inserted into a body opening of a mammal, preferably a human being, and subsequently said body opening is closed at least partially by way of a suture, crimping and/or glueing.

Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows individual elements as well as the process of preparing an implantable device, wherein in a) the pouch and a separate non-degradable implantable device are shown; in b) the pouch is shown and a non-degradable implantable device with two separate layers of material comprising a therapeutically active substance; in c) a non-degradable implantable device with the two separate layers inserted into the pouch; in d) the pouch after closing the opening thereof; in e) a schematic representation of the release of the therapeutically active substance out of the implantable device

FIG. 2 shows stress-strain curve of a tensile test conducted on bacterial cellulose.

### DESCRIPTION OF PREFERRED EMBODIMENTS

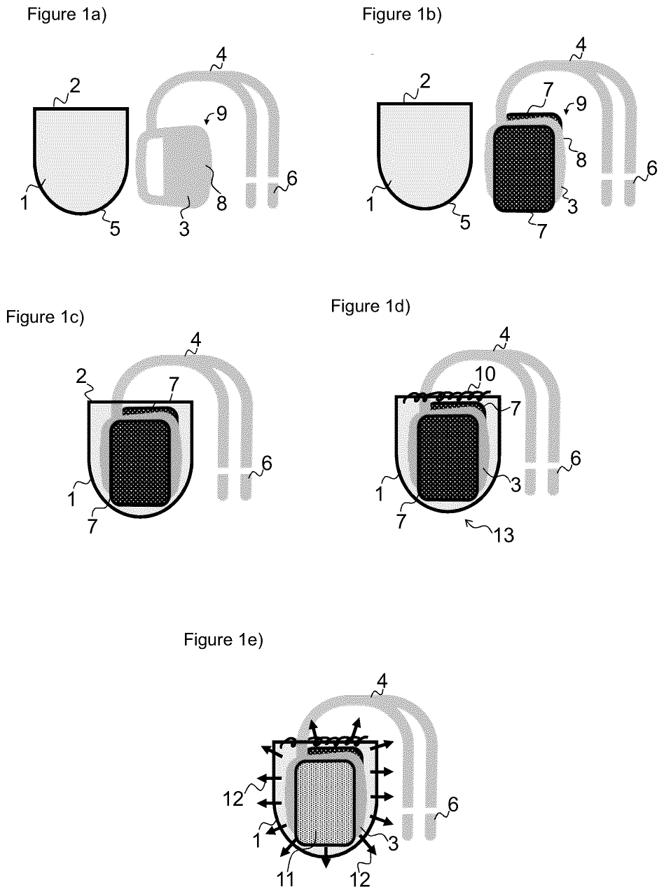

FIG. 1 schematically shows the method as claimed using an example of a pacemaker. A cellulose pouch 1, the production of which will be detailed further below, is provided in a wet or humid form as a separate element as illustrated in FIG. 1a). It is typically taken out of a package providing for wet storage by the corresponding personnel. Tools can be used for handing these cellulose pouches, to simplify the procedure. The pouch is based on a patch which is typically folded over one edge and has a sealed edge 5 and only one opening 2 thereby forming the actual pouch for insertion of the non-degradable implantable device.

Separately the non-degradable implantable device 3 is provided. In this case it is a pacemaker having an actual core element with front surface 8 and back surface 9, so it is a flat almost rectangular device. It further comprises wiring 4 which may be attached to an external device 6. However the proposed method may also be carried out with implantable device which do not have wiring or tubing to the outside, such as for example breast implants or the like.

Before inserting, as illustrated in FIG. 1b), the front surface 8 and the back surface 9 of the non-degradable implantable device 3 is covered by controlled release layers or pads 7 and 7′. These controlled release pads 7 are provided as separate elements and are typically unpacked from corresponding dry storage packages. As mentioned above, typically pouch and these controlled release pads are provided in separate packages or in separate compartments of one larger package as a kit of parts for ease of use. The making of these release pads 7 will be detailed further below. They may manually be arranged in contact with and facing the front and back surface of the non-degradable implantable device, and this may be assisted, in particular in case of very thin dry controlled release pads 7 by electrostatic attraction between the device 3 and the respective sheets of the release pads 7.

This assembly of the non-degradable implantable device 3 and the 2 controlled release pads 7, 7′ is now taken and insert into the pouch 1 through the opening, and the result of that process is illustrated in FIG. 1c). After that typically the opening is closed, in this case by way of a suture 10, as this is illustrated in FIG. 1d).

This prepared implantable device is then, subsequent to this preparation method, implanted into the human body. Under the conditions in the human body (temperature, physiological liquids) the therapeutically active substance starts diffusing/migrating through the porous cellulose patch material into the surrounding body tissue. If the therapeutically active substance is an antibiotic, it is then accordingly, over a defined period of time (sustained-release) released into that portion of the tissue where the risk of an infection is highest. So it is a very targeted selective release of the active agent exactly to the place and over the time as needed. Correspondingly an as low as possible dosage of an antibiotic can be used.

In this process, which is illustrated in FIG. 1e), the sheets 7 either just release the active ingredient, or there is a whole, if they are degradable, or dissolvable, at the same time as releasing the active ingredient start to disintegrate (partially degraded element illustrated as 11) and, after a certain time, vanish completely. Until then the release 12 of the active ingredient takes place.

### Manufacturing of the Pouch:

### Growth of the Cellulose Layer on the Mold:

### Materials:

| Name | Concentration |
|---|---|
| Acetobacter Xylinum¹ | 1 ml for 20 ml of medium |
| Growth Medium | 1x |
| Silicone mold | |

¹A. Xylinum belongs to the family of bacteria that ferment carbohydrates to vinegar and is commonly found in soil and decaying fruit. It is peculiar for its cellulose production. Other cellulose producing bacteria can be used.

### Procedure:

### Medium Composition and Preparation

| Elements | Amount | |
|---|---|---|
| KH₂PO₄ | 7000 | mg |
| (MgSO₄)7H₂O | 2130 | mg |
| H₃BO₃ | 4.3 | mg |
| Nicotinamide | 0.7 | mg |
| FeSO₄*7H₂O | 9.5 | mg |
| Na₂HPO₄*12H₂O | 3380 | mg |
| (NH₄)₂SO₄ | 3540 | mg |
| Ethanol | 4730 | mg |
| Distilled Water | 1 | liter |

The resulting solution is autoclaved for 30 minutes at 121° C. After cooling down to room temperature, 50 ml of a filtered Glucose solution (50% in distilled water) is added.

Set-up of the bioreactor:
Mix homogenized cellulose (containing bacteria) and medium in a 1:20 ratio within a sterile container.
Place the mix in contact with the silicone mold taking care that no air bubbles are trapped at the interface.
The bioreactor is carefully placed in the incubator and maintained at 29° C., in a humidified environment (70% humidity).
Let bacteria grow for a sufficient time. The incubation time is proportional to the desired total thickness of the resulting cellulose layer. The following table provides some experimental indication of this correlation:

| Incubation time | Final layer thickness |
|---|---|
| 24 h | 2 mm |
| 48 h | 3 mm |
| 96 h | 4 mm or thicker |

Harvesting of the cellulose layer:
At the end of the fermentation period the silicone mold and the cellulose layer are removed from the incubator.
After separating the cellulose layer from the silicone mold, the cellulose layer is immersed into a NaOH (1M in distilled water) solution at room temperature
The cellulose layer is carefully peeled off the silicone mold within the solution.
The NaOH solution with the cellulose layer is then kept at 80° C. in an oven for 80 minutes (i.e. the whole layer is immersed in the NaOH solution; prior to that, bacteria and medium are trapped within and at the surrounding of the cellulose; this step removes/washes (or “anneals”) the bacteria; the next steps are to replace NaOH with water).
The NaOH solution is then removed and distilled water is added.
The cellulose layer is washed with fresh distilled water 4 times for 1 h each to remove media residues.
The cellulose layer (which has the character of a hydrogel) is then washed with a 95% ethanol solution and then stored in ethanol until use.

This procedure yields a semi-transparent cellulose layer (in the visible spectrum). To improve transparency (up to 90% of incident light) longer incubation in ethanol (up to 1 week) can be used.

The resultant structures are stable upon dehydration/rehydration.

The cellulose layer is natively shaped as a pouch, therefore no further processing is required, however it is also possible to first produce a sheet and then form a corresponding pouch from that sheet.

Making of 3D-topographically structured elements:
The surface topography is imprinted during the molding process itself (with e.g. PDMS) or applied as additional layer (e.g. by gluing with the PDMS itself an already structured layer, as previously described). The silicone mold has a thickness between 0.5-2 mm and an inner cavity of the shape, sizes and dimensions of the object to be covered with cellulose. The silicone mold can feature an additional layer at its top for facilitating its placement into the bacterial culture.

The silicone mold with surface topography is placed in the bacterial culture so to allow for:
complete wetting of the silicone mold external surface
air filling of the internal cavity.

The placement of the silicone mold can be helped with a bioreactor consisting of two chambers, for air and bacteria in medium, respectively. Oxygen circulation within the air chamber can be facilitated by leaving the chamber open or by controlling the oxygen flow in it, by using e.g. a pump or a gas bottle with a system of valves.

After the culturing time, a cellulose layer is formed at the mold interface. The cellulose pouch/cover/pocket features surface topography on its internal surface and can easily be removed, washed, processed and sterilized as previously described for the flat cellulose patches. The cellulose pocket is eventually flipped inside-out in order to feature surface topography on its external surface. The target object can eventually be inserted within the cellulose pocket. The enclosing of the object can be optimized by suturing the open side of the cellulose pocket.

### Manufacturing of the Controlled Release Pad:

Minocycline and Rifampin (as therapeutically active substances or APIs) implantable films are produced as follows:

### Active Blend Mixing and Coating Procedure:

1. APIs shall be dissolved in a suitable solvent or mix of suitable solvents together with a polymeric carrier material, in the form of at least one degradable or non-degradable polymeric material as listed above, wherein typically organic solvents are used. A plasticizer can be added, in particular to tune viscosity of the mixture.
2. The components shall be mixed until they are fully dissolved.
3. Once mixed, the solution shall be coated to the a thickness between 10 and 3000 μm, preferably between 10 and 50 μm.
4. The layer formed during the coating procedure shall be dried at ambient conditions or elevated temperature (or vacuum if needed).
5. The final elements can be cut to measure from the resulting dry layer, for example using a die cutting process.

To show that the wet BC does not show important elastic characteristics, as is the case with elastomers, the tensile loading-unloading curve was analyzed. During tensile testing, the stress-strain curve is characterized by an elastic and a plastic region prior to rupture. When a load is applied to a material, it elongates. The strain thus becomes greater than 0. The elastic region is characterized by a return to a null strain-state when the load is removed, whereas the plastic region maintains a non-zero strain after unloading (see. e.g. David Roylance. 3.11 Mechanics of Materials. Fall 1999. Massachusetts Institute of Technology: MIT OpenCourseWare, https://ocw.mit.edu. License: Creative Commons BY-NC-SA).

FIG. 5 given in Kehao Wang, Barbara K. Pierscionek, Biomechanics of the human lens and accommodative system: Functional relevance to physiological states, Progress in Retinal and Eye Research, Volume 71, 2019, Pages 114-131, ISSN 1350-9462, https://doi.org/10.1016/j.preteyeres.2018.11.004. shows examples of loading and unloading stress-strain curves of (a) a linear elastic model (b) a non-linear elastic model and (c) a plastic model. (& is the strain, σ is the stress).

FIG. 2 shows the stress-strain curve of a tensile test conducted on biosynthesized cellulose, where the loading and unloading is reported at multiple loads. It is clearly visible that after applying and releasing tension to the blosynthesized cellulose, it is unable to go back to its initial strain-free state, even at very low loads.

LIST OF REFERENCE SIGNS

| | |
|---|---|
| 1 | cellulose pouch |
| 2 | opening of 1 |
| 3 | non-degradable implantable device |
| 4 | wiring/tubing to 3 |
| 5 | sealed edge of 1 |
| 6 | external device of 3 |
| 7 | controlled release pad |
| 8 | front face of 3 |
| 9 | backside face of 3 |
| 10 | suture |
| 11 | partially degraded/dissolved 7 |
| 12 | release of active agent, antibiotic |
| 13 | ready to implant implantable device |

1. Method for the preparation of an implantable device,
wherein in a first step a non-degradable implantable device is at least partially covered or surrounded by at least one separate layer of material comprising a therapeutically active substance providing for release of said substance under physiological conditions, including sustained controlled release of said substance under physiological conditions,
and wherein in a second step this is inserted into a wet or humid pouch of at least one of self-supporting hydrogel, cellulose or collagen through an opening thereof.

2. Method according to claim 1, wherein the wet or humid pouch has a water content in the range of 50-98%.

3. Method according to claim 1, wherein the wet or humid pouch has a thickness of at least 0.3 mm,
and/or the wet or humid pouch has, at least on its outside surface, a topographical surface structure with a height in the range of 0.5-2 μm, and in case of a groove/ridge topographical structure a periodicity of the structure in the range of 0.5-100 μm and in case of a pillar topographical structure a periodicity of the structure a periodicity at least in one dimension, or in three different directions, in the range of 5-50 μm.

4. Method according to claim 1, wherein the wet or humid pouch is a cellulose pouch and is produced before said first step using self-assembled production of a topographically surface structured cellulose element wherein
a mold with on one side a first surface which is in a complementary manner topographically surface structured and which is permeable to oxygen is provided, wherein a liquid growth medium containing cellulose producing bacteria is provided, and wherein the mold is placed to form a liquid/air interface of the liquid growth medium such that the side of the mold with the first surface is in direct contact with the liquid growth medium, and with an opposite side is facing air or a specifically provided oxygen containing gas surrounding, allowing for said bacteria to produce and deposit cellulose on said first surface and developing on the interface therewith a topographically surface structured surface complementary thereto, until a contiguous cellulose layer with a thickness of the element of at least 0.3 mm is formed;
and wherein in a following step the element is removed from said mold, and said pouch is produced from said element,
wherein between this production and the first step the element or the pouch can be stored in a wet environment, including in a corresponding suitable and adapted container.

5. Method according to claim 1, wherein said separate layer comprises, as therapeutically active substance, at least one antibiotic, antiseptic haemostatic, antinflammatory, chemotherapic, hormone, chemokine or disinfectant or a combination thereof.

6. Method according to claim 5, wherein the concentration of the therapeutically active substance in the layer is in the range of 5-50%, given as % w/w dry with respect to the total of the layer.

7. Method according to claim 1, wherein said layer is provided as at least 2 sheets which are put on opposing main faces of the non-degradable implantable device before insertion into said wet or humid pouch.

8. Method according to claim 1, wherein said layer has a thickness in the range of 0.01-3 mm.

9. Method according to claim 1, wherein said non-degradable implantable device is selected from the group of cardiovascular implant and/or device, including a pacemaker; cardioverter defibrillator; neurostimulator, neuromodulator, implantable pulse generator; cosmetic implant, including in the form of a breast implant, cuff implant, pectoral implant, biceps implant, buttock implant, gluteal implant; orthopaedic prosthesis; a sensor and/or electrical stimulation device; draining system, including a catheter; pump or tubing system;
ophthalmological device; hearing device; bionic device.

10. Method according to claim 1,
wherein subsequent to the second step in a third step said opening is closed, including by way of a suture, crimping or glueing, wherein elements, including tubing and/or wiring attached to and connected with said non-degradable implantable device if present remain penetrating said opening.

11. Kit of parts for use in a method according to claim 1, comprising
at least one wet or humid pouch having an opening in a first wet package;
and at least one layer of material comprising a therapeutically active substance providing for sustained controlled release of said substance under physiological conditions in a separate wet or dry package.

12. Kit of parts according to claim 11, wherein there is provided at least two layers in said separate package.

13. Kit of parts according to claim 11, wherein said first package comprises elements for maintaining controlled humidity, including in the form of aluminium or aluminium plastic laminate pouches,
and/or wherein said separate package comprises elements for maintaining dryness, including desiccant elements.

14. Implantable device producible or produced according to claim 1, wherein a non-degradable implantable device at least partially covered or surrounded by at least one separate layer of material comprising a therapeutically active substance providing for sustained controlled release of said substance under physiological conditions, is located in a wet or humid pouch.

15. Method of implanting an implantable device according to claim 14 into a human or animal body, wherein said implantable device is inserted into a body opening of a mammal, including a human being, and subsequently said body opening is closed at least partially by way of a suture, crimping and/or glueing.

16. Method according to claim 1, wherein the wet or humid pouch, in the form of a cellulose pouch, has a water content in the range of 90-98%.

17. Method according to claim 1, wherein the wet or humid pouch, in the form of a cellulose pouch, has a thickness in the range of 0.5-5 mm,
and/or the wet or humid pouch, in the form of a cellulose pouch, has, at least on its outside surface, a topographical surface structure with a height in the range of 0.5-2 μm, and in case of a groove/ridge topographical structure a periodicity of the structure in the range of 0.5-100 μm and in case of a pillar topographical structure a periodicity of the structure a periodicity at least in three different directions, in the range of 7-15 μm.

18. Method according to claim 1, wherein said separate layer comprises, as therapeutically active substance, at least one antibiotic, antiseptic haemostatic, antinflammatory, chemotherapic, hormone, chemokine or disinfectant or a combination thereof, wherein the therapeutically active substance is selected from at least one antibiotic selected from the group of tetracyclines, penicillins, macrolides, ansamycines, including Tetracycline, Chlortetracycline, Oxytetracycline, Demeclocycline, Lymecycline, Meclocycline, Methacycline, Minocycline, Rolitetracycline, Doxycycline, Tigecycline, Eravacycline, Sarecycline, Omadacycline, Rifampicine, or a combination thereof.

19. Method according to claim 5, wherein the concentration of the therapeutically active substance in the layer is in the range of 25-40%, given as % w/w dry with respect to the total of the layer.

20. Method according to claim 5, wherein in addition to the therapeutically active substance the layer comprises at least one degradable or non-degradable polymeric material, including in the form of a degradable polymeric material selected from the group consisting of polylactic acid, poly(lactic-co-glycolic acid) (PLGA), polyglycolic acid, poly(L-lactide) (PLLA), poly(D.L-lactide), (PLA) polyglycolic acid polyglycolide (PGA), poly(L-lactide-co-D.L-lactide) (PLLA/PLA), poly(D, L-lactide-co-glycolide) (PLA/PGA), poly(glycolide-co-trimethylene carbonate) (PGA/PTMC), poly(D.L-lactide-co-caprolactone) (PLA/PCL), poly(glycolide-co-caprolactone) (PGA/PCL), poly(oxa) esters, polyethylene oxide (PEO), polydioxanone (PDS), polypropylene fumarate, polyethyl glutamate-co-glutamic acid), poly(tert-butyloxy-carbonylmethyl glutamate), poly caprolactone (PCL), polycaprolactone co-butylacrylate, polyhydroxybutyrate (PHBT), polyhydroxybutyrate, poly(phosphacene), poly(phosphate ester), poly(amino acid), polydepsipeptides, polyiminocarbonates, poly(dimethyl-trimethylene carbonate)-co-(trimethylene carbonate), poly(orthoesters), tyrosine-derived polycarbonates, tyrosine-derived polyiminocarbonates, tyrosine-derived polyphosphonates, polyethylene oxide, polyethylene glycol, polyalkylene oxides, and hydroxypropylmethylcellulose polysaccharides including hyaluronic acid, chitosan and regenerate cellulose, and proteins including gelatin and collagen, and mixtures and copolymers thereof.

21. Method according to claim 5, wherein in addition to the therapeutically active substance the layer comprises at least one degradable polymeric material, wherein a mixture of poly(lactic-co-glycolic acid) (PLGA) and polyethylene glycol is used.

22. Method according to claim 1, wherein said layer is provided as at least 2 sheets which are put on opposing main faces of the non-degradable implantable device before insertion into said wet or humid pouch, in the form of a is a cellulose pouch and wherein said layer is provided as a dry layer with a water content of less than 5%, or less than 2%, or less than 1%, in each case given as % w/w.

23. Method according to claim 1, wherein said layer has a thickness in the range of 0.02-0.2 mm.

24. Kit of parts according to claim 11, comprising
at least one wet or humid pouch, in the form of a pouch of at least one of self-supporting hydrogel, cellulose or collagen, having an opening in a first wet package;
and at least one layer of material comprising a therapeutically active substance providing for sustained controlled release of said substance under physiological conditions in a separate wet or dry package.

25. Kit of parts according to claim 11, wherein there is provided at least two layers in said separate package, and wherein the layers take the form of rectangular or quadratic sheets with length and/or width in the range of 50-110 mm, or in the range of 60-90 mm,
or wherein they take the form of a rectangle having a length in the range of 70-110 mm, or in the range of 80-90 mm, and a width in the range of 50-90 mm, or in the range of 60-80 mm.

26. Implantable device according to claim 14, wherein a non-degradable implantable device at least partially covered or surrounded by at least one separate layer of material comprising a therapeutically active substance providing for sustained controlled release of said substance under physiological conditions, is located in a wet or humid pouch, in the form of a pouch of at least one of self-supporting hydrogel, cellulose or collagen.
